(19) United States
(12) Patent Application Publication
(10) Pub. No.: US 2004/0209813 A1
(43) Pub. Date: Oct. 21, 2004

(54) Caspase inhibitors for inhibiting blood cell proliferation and for treating autoimmune diseases

(76) Inventors: Ralph C. Budd, South Burlington, VT (US); Jurg Tschopp, Epalinges (CH); Takao Kataoka, Shinjuku-ku (JP)

Correspondence Address: HESLIN ROTHENBERG FARLEY & MESITI PC, 5 COLUMBIA CIRCLE, ALBANY, NY, 12203, US

(21) Appl. No.: 10/829,303

(22) Filed: Apr. 21, 2004

Related U.S. Application Data
(63) Continuation of application No. 09/973,476, filed on Oct. 9, 2001.
Application No. 09/973,476 is a continuation of application No. PCT/EP00/03019, filed on Apr. 5, 2000.

(30) Foreign Application Priority Data
Apr. 6, 1999 (DE) 199 15 465.1

(51) Int. Cl. A61K 38/17

(52) U.S. Cl. 514/12

(57) ABSTRACT

The use of inhibitors of cysteine aspartate proteins (caspases) for treating diseases or disorders caused by a hyperproliferation of peripheral blood lymphocytes is disclosed. The use of caspase inhibitors for suppressing the immune system is also disclosed.

### CROSS REFERENCE TO RELATED APPLICATION AND CLAIM FOR FOREIGN PRIORITY

This application is a continuation of co-pending International Patent Application Number PCT/EP00/03019, filed Apr. 5, 2000, and claims priority from DE Patent Application Number 199 15 465.1 filed Apr. 6, 1999. The entire disclosures of the prior applications are incorporated herein by reference.

### FIELD OF THE INVENTION

The present invention relates to the use of inhibitors of cysteine aspartate proteins (caspases), which have an important function in intracellular signal transduction, to inhibit the proliferation of cells. The invention also relates to the use of one or more caspase inhibitors to treat diseases, disorders or pathophysiological states that are based aetiologically on a hyperproliferation of lymphocytes and to the use of one or more caspase inhibitors to suppress the immune response of lymphocytes.

### BACKGROUND OF THE INVENTION

It is known from the literature that caspases (cysteine aspartate proteins) are of considerable importance for the intracellular signal transduction of certain apoptosis-stimulating signals. Apoptosis is physiologically finely regulated targeted cellular death, which can be induced, for example, by binding ligands to receptors, e.g. TNF binding or CD95 (Fas) binding, by withdrawing defined growth factors, by removal from an extracellular matrix, by ionizing radiation, staurosporin or even through glucocortocoids. It is also known from the literature that, in particular after an oligomerization of the Fas receptor (CD 95) induced by extracellular sFasL, i.e., by a soluble ligand of the Fas receptor, a cascade of proteolytic reactions is triggered which finally ends in cell apoptosis. Here, each subsequent caspase is activated through proteolytic cleavage in accordance with the caspase sequence in the cascade. A central function in this apoptotic signal transduction is taken up at the starting point of the proteolytic cascade by the caspase referred to as “caspase-8”, which belongs to the DISC complex and which settles on the Fas receptor through the linker protein FADD. In the DISC complex the procaspase-8, which is still inactive enzymatically, is cleaved into two subsequent reaction steps, through which active caspase-8 is formed, and which dissociates from the DISC complex as a heterotetramer. The active caspase-8 can now, for example, activate the caspases which are still functioning distally in the cascade sequence, namely caspase-3 or caspase-7.

It has also been written that different types of caspase inhibitors exist which block the apoptotic signal transduction path. Here, non-biologically occurring caspase inhibitors are to be differentiated from biological, e.g. viral caspase inhibitors.

Up to now the literature has shown exclusively that caspases become important in connection with apoptotic signal transduction. Consequently, caspase inhibitors, whether they have natural or non-natural origins, have also only been assigned a function in the inhibition of apoptosis. The state of the art does not describe other functional characteristics of caspases in intracellular metabolism, and therefore other possibilities for using caspase inhibitors.

The present invention is based on the object of finding other functional cell-physiological activities for individual caspases and therefore determining new possibilities for using caspase inhibitors, including under pathophysiological aspects.

### SUMMARY OF THE INVENTION

In accordance with one embodiment of the invention, a caspase inhibitor alone, or in combination with one or more other caspase inhibitors, may be used to inhibit the proliferation of peripheral blood lymphocytes (hereinafter, “PBL”). This is believed to be based on a finding that caspases not only participate in the transmission of exogenous apoptotic signals, but that they can also take over functions in the proliferation of PBL in accordance with corresponding exogenous stimulation. This is thought to be due to the fact that two stimulating signals are required for the proliferation of PBL. Together with the stimulating binding reaction at the T-cell receptor CD3 complex, which has been known for some time, there must also be a further co-stimulating signal. This co-stimulus is the binding of extracellular FasL to Fas with subsequent intracellular signal transduction through the activation of caspases. In this way, extracellular FasL has a co-stimulating effect for example together with the stimulation of T-cells at the T-cell receptor (TCR)/CD3 complex.

Caspases therefore have a twin function, namely, on the one hand, as cascade members of apoptotic signal transduction and, on the other hand, in accordance with the present invention as intracellular elements of co-stimulating exogenous signals for PBL proliferation as well. Inhibition of the caspases leads therefore in accordance with the present invention to inhibition of PBL proliferation. This is why caspases also have an effect as inhibitors of the proliferation of lymphocytes and here in particular of PBL. In accordance with the present invention, therefore, caspase inhibitors can inhibit the)e proliferation of B and in particular of T-lymphocytes, above all with lymphocytes circulating in the cardiovascular system.

The caspase inhibitors used to inhibit proliferation can exercise their inhibitory effect through reversible or irreversible inhibition of a caspase or of several caspases. Intracellular signal transmission is blocked in this way.

Caspase inhibitors which inhibit the function of caspase-8 are of particular interest in the present invention. Caspase-8 inhibition can be necessitated, for example, by substances which prevent the cleaving of the prodomain of the procaspase-8. Thus, an active caspase-8 fraction cannot be formed which would permit the further transduction of the signal. It is also conceivable through the use of caspase inhibitors of this type which block the enzymatic activity of the proteolytically cleaved, and therefore active, caspase-8 fraction. This is possible, for example, through binding to the active center of the caspase-8.

A further embodiment of the present invention is based on the finding that caspase inhibitors can be used to treat diseases, disorders or pathophysiological conditions or to serve as an active ingredient in a medicament that can be used to treat diseases, disorders or pathophysiological conditions of types which are caused by a hyperproliferation of PBL. In accordance with the principles or the present invention, because co-stimulation through FasL/FasR (which leads to activation of one or more caspases) is necessary for the proliferation of PBL, it is possible with the help of caspase inhibitors to prevent pathophysiological proliferation of PBL. The use of caspase inhibitors can therefore be indicated in patients who have tumor diseases of the lymphatic system. The use of caspase inhibitors is particularly advantageous if the tumor disease is based on hyperproliferation of degenerate T- or B-lymphocytes. The caspase inhibitor(s) may be utilized to produce a medicament for the treatment of diseases of this kind.

The use of caspase inhibitors is also indicated to suppress an excessive immune response, whether through B-lymphocytes or through T-lymphocytes. Here in particular the use of caspase inhibitors should be noted to treat diseases, disorders or pathophysiological conditions in which the immune defenses are directed against the body's own structures. For this reason, the use of caspase inhibitors to combat autoimmune diseases is preferred.

In accordance with the principles of the present invention, one or more caspase inhibitors may be utilized to produce a medicament for the treatment of such autoimmune diseases, disorders or pathophysiological conditions as rheumatoid arthritis, systemic Lupus erythematosis, diabetes mellitus or multiple sclerosis, for example.

In addition, the use of a caspase inhibitor or the combined use of several caspase inhibitors is also advantageous if a fundamental suppression of the immune system is desired. Here, caspase inhibitors are particularly suitable for suppressing the immune response supported by peripheral blood lymphocytes (PBL). Wide-ranging immune suppression is indicated above all, following transplantations of allogenic cells, tissue or organs. The transplant patient's rejection response to foreign cells, foreign tissue or foreign organs can be repressed through the use of caspase inhibitors or by their use to produce a medicament, without serious side effects being expected.

Caspase inhibitors may be substances which occur naturally and may also already have a physiological effect as caspase inhibitors. However, caspase inhibitors may also be, for example, organic-chemical molecule structures or short non-natural peptides. In general, nonbiologically occurring substances or molecules which inhibit caspases are preferred.

Oligopeptides or polypeptides which can block caspases as inhibitors are preferred. Oligopeptides with 3 to 15 amino acid chain lengths are particularly suitable, those with 3 to 6 amino acid chain lengths are especially preferred, and tetrapeptides are particularly advantageous. The oligopeptides may be part sequences of naturally occurring proteins which may also have a caspase inhibitory effect. For example, part sequences of the bacterial protein CrmA can be used. Caspase inhibitors of this type on a peptide base can be modified chemically at reactive groups of the amino acid side-chains, such as, for example, at an amino or carboxy group, or at the respective N— or C-terminus of the peptide. In this way, for example the stability of the peptide-based inhibitor can be increased or the passage of the inhibitor through the cell membrane can be made easier.

Preferred modifications at the C-terminus of the oligopeptide or polypeptide include, but are not limited to, aldehyde derivatization and the introduction of a ketone group, such as a fluoromethylketone or an acyloxymethylketone group.

Peptides that contain the amino acid sequences VAD, IETD or YVAD (single-letter code) are particularly preferred for use as caspase inhibitors. In addition, chemical modifications of the peptides VAD, IETD or YVAD, are also suitable for use in accordance with the present invention. Particularly preferred for use as caspase inhibitors to inhibit proliferation are peptides modified by a fluoromethylketone (“fik”) group at the C-terminus, such as, for example, IETD-fmk, zVAD-fmk or YVAD-fmk.

Along with non-natural molecule structures for use as inhibitors of cell proliferation, the present invention also covers those biologically occurring substances, and in particular, peptides or proteins, which prove physiologically to be effective caspase inhibitors. These may, for example, be substances of viral, bacterial or eukaryotic origin. The bacterial protein CrmA might be referred to as an example.

Depending on the indication, the caspase inhibitor or inhibitor(s) can be administered either systemically, e.g., orally, intravenously, interperitoneally or intramuscularly, or topically. In some instances, the use of adjuvants may be necessary to produce a particular dosage form. The specific galenic preparation will depend on the respective indication area and desired route of administration.

### BRIEF DESCRIPTION OF THE DRAWINGS

The present invention will be explained in detail by reference to the accompanying Figures, wherein:

FIG. 1 shows the effect of different caspase inhibitors on human T-cells. For this purpose the T-cells were stimulated with 10 μg/ml soluble anti-CD3 antibodies in the presence of various caspase inhibitors. The inhibitors IETD-fmk and zVAD-fmk (caspase-8 inhibitors), YVAD-fmk (caspase-1 inhibitor) were used, as well as DMSO for control purposes in comparable concentrations in each case. Plotted, the proliferation of the stimulated T-cells (cpm, measured by [³H] thymidine incorporation) is found as a function of increasing concentrations of caspase inhibitors. In particular, with concentrations above 25 μM, the caspase-8 inhibitors, IETD-fmk and zVAD-fmk, display a clear inhibiting effect on cell proliferation.

FIG. 2 shows the effects that result from an addition of FasFc or IgG for the proliferation of T-lymphocytes. Here, the T-lymphocytes were activated with the help of immobilized anti-CD3 antibodies (0.5 μg/ml). Whereas the dose-dependent addition of FaS-Fc clearly reduces the number of cells measured after the expiry of three days (measured here with cpm), the addition of IgG has no effect at all on cell proliferation. The result in FIG. 2 is thus consistent with the model which postulates that the immobilised FasL inhibitor Fas-Fc blocks the co-stimulating signal which is necessary for proliferation, namely FasL/Fas binding.

FIG. 3 shows the specific proliferation inhibiting effects of three caspase inhibitors, YVAD-fmk, zVAD-fmk and IETD-fmk. For this purpose, PBL were cultivated in each case with the caspase inhibitors referred to above and then were stimulated (50 ng/ml) with 3 μm/ml soluble anti-CD3 antibodies and crosslinked sFasL. Cross-linked sFasL represents oligomerized sFasL. The sFasL carries a FLAG sequence to which the crosswise networking anti-FLAG antibodies bind, which leads to oligomerizing. Similar to what was shown in FIG. 1, the caspase-8 inhibitors prove to be the most effective T-cell proliferation inhibitors even with combined stimulation with anti-CD3 antibodies and FasL.

FIG. 4 illustrates the correlation of IL-2 expression and the use of caspase inhibitors using a bar diagram. On the one hand, it can be seen that the combined stimulation of anti-CD3 antibodies and FasL shows significantly increased IL-2 production as a consequence, as compared with activation carried out solely with anti-CD3 antibodies (referred herein as control). On the other hand, with anti-CD3 antibody stimulation, the caspase-8 inhibitors IETD-fmk and zVAD-fmk prove here as well to be particularly effective with regard to the suppression of IL-2 production. The findings shown in FIG. 4 are based on experiments in which 10⁶ PBL/ml were cultivated with immobilized anti-CD3 antibody (3 μg/ml) and FasL (50 ng/ml) in the presence or absence of the caspase inhibitors referred to above (50 μM). The supernatants were removed after 24 hours and examined with the help of a CTLL bioassay for their IL-2 concentration.

FIG. 5 shows that the inhibition of T-cell proliferation of caspase-8 inhibitors, using zVAD-fmk as an example, can be cancelled by adding IL-2. This means that the activity of caspase-8 is significant for the IL-2 production of T-cells. In the experiments on which FIG. 5 is based, PBL were activated with 10 μg/ml soluble anti-CD3 antibody in the presence or absence of zVAD-fmk (50 μM) and in the presence of anti-CD3 antibody zVAD-fmk and 500 U/ml recombinant human IL-2, respectively. While in accordance with the invention, the addition of zVAD-fmk has the effect of a clear reduction in the number of cells as against the control at the time of observation, cell proliferation increases dramatically with the addition of IL-2.

FIG. 6 provides a Western blot. Human T-cells were cultivated either without stimulation (control), with soluble anti-CD3 antibody alone (3 μg/ml), or with anti-CD3 antibody and sFasL (50 ng/ml) which, as described above, is crosslinked via its FLAG sequence. The cell lysates were examined in accordance with the times shown in FIG. 6, with regard to the expression of procaspase-8 or of cleavage products of the procaspase-8. The black arrow indicates the position of the enzymatically inactive procaspase-8 in the Western blot, while the open arrow indicates the enzymatically active, proteolytically cleaved 26 kDA fragment.

FIG. 6 shows clearly that the highest concentration of active caspase-8 in the cell lysates is found four hours after the start of cultivation. Here, the test approach with combined cell stimulation through anti-CD3 antibody and FasL shows a significantly increased active caspase-8 fraction as against the T-cell stimulation which was brought about solely through anti-CD3 antibody. In a further experimental approach, T-cells were stimulated with a combination of anti-CD3 antibodies and FasL for a period of 6 hours, namely in the presence of 50 μM of the caspase-8 inhibitor IETD-fmk. Due to the effects of the caspase-8 inhibitor, the cleavage of caspase-8 during the stimulation was blocked, and a 26 kDa fraction cannot be detected in the Western blot in this case.

### DETAILED DESCRIPTION OF THE INVENTION

The principles of the present invention are explained in further detail by means of the following examples.

### EXAMPLE 1

To verify the activity of caspase inhibitors as inhibitors of cell proliferation their effect on human peripheral blood lymphocytes was examined.

For this purpose, the latter were prepared through Ficoll-Hypaque centrifugation. The cells (5×10⁴ cells per well) were then cultivated on 96-well plates in the presence of different caspase inhibitors or, for control purposes, in their absence. The concentration of the caspase inhibitors was varied in a range of 25 to 100 μM. Finally, the cells were stimulated through anti-CD3 antibodies (TR66) or through a combined stimulation with anti-CD3 antibodies and soluble recombinant FasL with or without anti-FLAG sequence antibodies 1 μg/ml.

Cell proliferation was measured during the last 18 hours of the four-day cultivation period. The measuring variable for cell proliferation was the incorporation of [³H] thymidine into the proliferating cells.

The caspase inhibitors which were used (YVAD-fmk, zVAD-fmk and IETD-fmk) were products from Bachem and Enzyme System Products. The recombinant FasL came from Alexis.

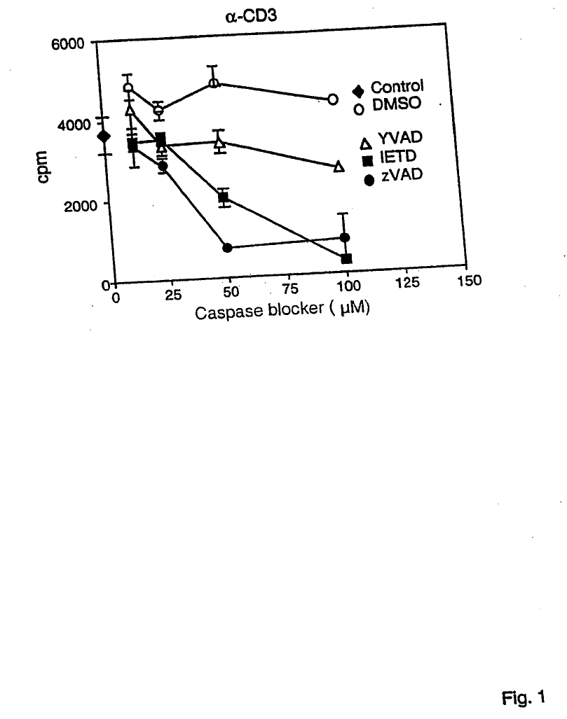

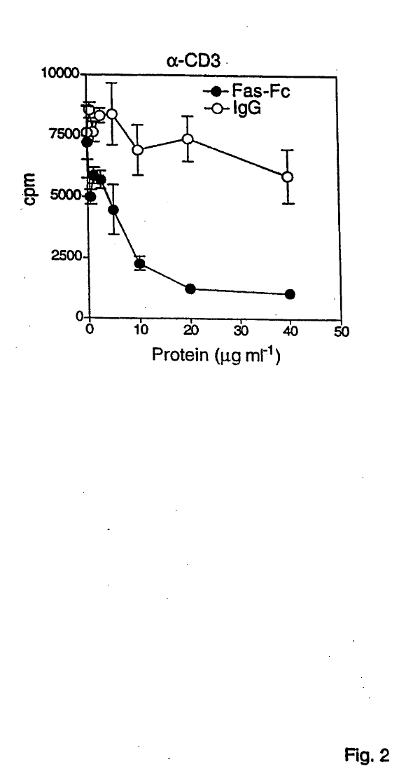

In the case of IETD-fmk and zVAD-fmk the caspase inhibitors which were used brought about complete inhibition of the stimulation brought about by the anti-CD3. The caspase inhibitor zVAD-fmk permits a partial inhibition of the cell proliferation to be detected (FIG. 1).

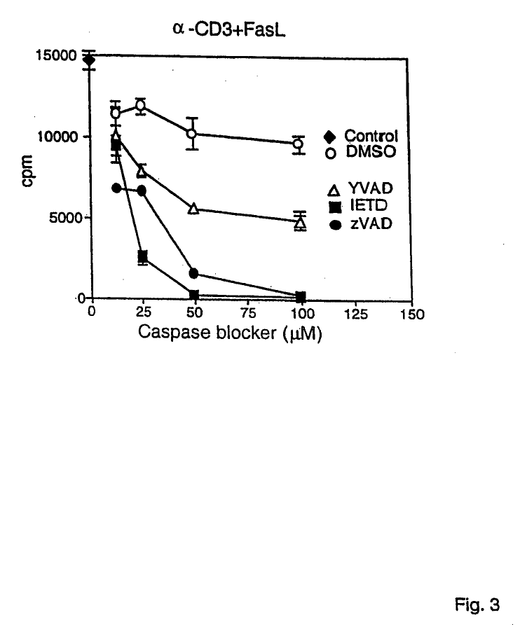

The combined stimulation of the human peripheral lymphocytes through anti-CD3 antibodies and FasL leads as well to similar results with regard to the activity of the caspase inhibitors which were used (FIG. 3).

### EXAMPLE 2

The inhibition of the proliferation in the fraction of the T-lymphocytes through caspase inhibitors was determined in a second experimental system.

For this purpose, T-lymphocytes (10⁶/ml) were stimulated with immobilized anti-CD3 antibody (3 μg/ml) with or without crosslinked FasL (50 ng/ml). The supernatants of these preparations with a caspase inhibitor or without a caspase inhibitor as control were removed after the stimulation period and their respective IL-2 concentration was measured using a CTTL bioassay. The IL-2 concentration in the supernatants reflects the T-cell proliferation.

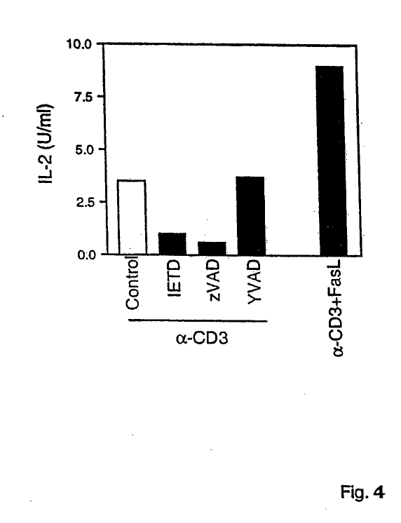

The results show that the presence of caspase inhibitors, particularly zVAD-fmk and IETD-fmk, blocks T-cell proliferation. In these test preparations, only weak IL-2 activity is seen (FIG. 4). Two test preparations without the addition of a caspase inhibitor serve as a measure for the assessment of the production of IL-2, which is to be expected after stimulation with anti-CD3 antibody or in combination with FasL (FIG. 4, bar left and right, respectively).

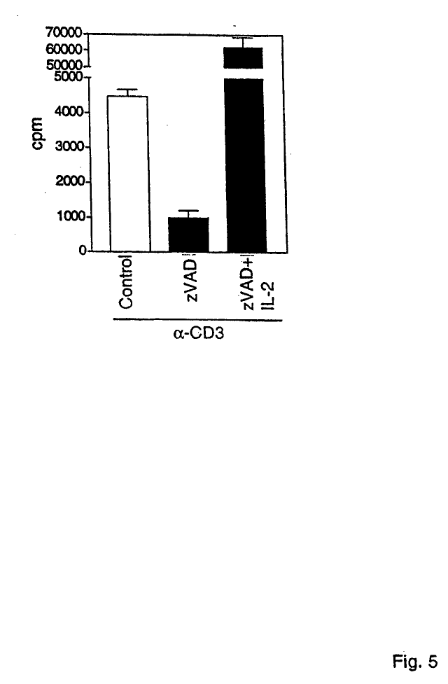

The inhibition of proliferation in these preparations with a caspase inhibitor can be cancelled out by adding exogenous recombinant IL-2. Greatly increased [³H] thymidine incorporation as against the control preparation can be measured in this case (FIG. 5).

### EXAMPLE 3

In this embodiment, inactive human T-lymphocytes were cultivated under different conditions: (i) as control without stimulation; (ii) stimulation with soluble anti-CD3 antibody (3 μg/ml) only; or (iii) combined stimulation with anti-CD3 antibody and sFasL (50 μg/ml, crosslinked through the FLAG/sequence “cross-linked”).

After 0, 2, 4, 6 and 22 hours, cells were taken from the different preparations and lysated. The cell lysates were examined with the Western blot technique with respect to their caspase-8 activity (procaspase-8, 55 kDa fraction, or proteolytically cleaved, active caspase-8, 26 kDa fraction, respectively).

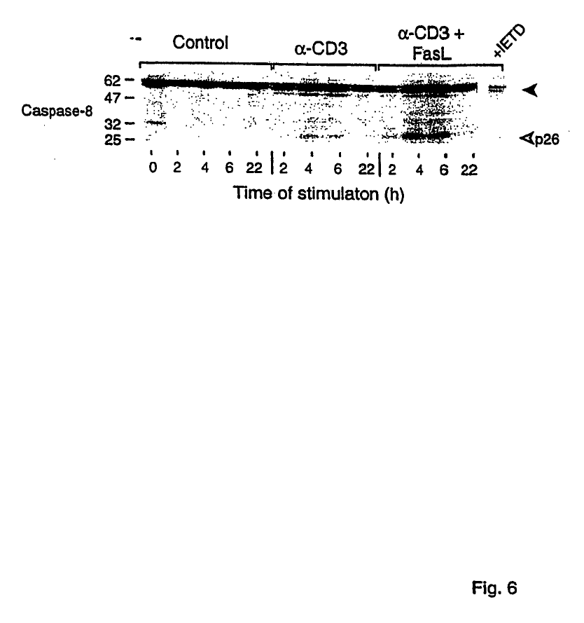

A band of the active 26 kDa fraction appears only after stimulation with anti-CD3 antibody or more intensively after combined stimulation with sFasL. The active 26 kDa caspase-8 fraction, which is created through proteolysis from the 55 kDa fraction, is found in its highest combination four hours after the start of stimulation (FIG. 6).

In a further test approach, inactive T-lymphocytes were stimulated in the presence of the caspase inhibitor IETD-fmk (50 μM) with anti-CD3 antibody and crosslinked sFasL (see above) and lysated 6 hours after the start of stimulation. In contrast to the test approach without a caspase inhibitor, the Western blot application does not lead to any 26 kDa band being detected (FIG. 6, right). This proves that the proteolytic cleavage of caspase-8 is also effectively blocked in the case of co-stimulation with sFasL through IETD-fmk.

We claim:

1. A method of suppressing the immune system of a human after allogenic cell, tissue or organ transplantation, the method comprising administering to the human a therapeutically effective amount of a caspase-8 inhibitor, or a pharmaceutically effective salt thereof.

2. The method of claim 1, wherein the caspase-8 inhibitor has a non-biologically occurring molecular structure.

3. The method of claim 1, wherein the caspase-8 inhibitor comprises an oligopeptide or polypeptide.

4. The method of claim 3, wherein the caspase-8 inhibitor comprises a tetrapeptide or a partial sequence of a native protein.

5. The method of claim 3, wherein the molecular structure of the oligopeptide or polypeptide includes a chemical modification at the N— or C-terminus.

6. The method of claim 5, wherein the chemical modification is at the C -terminus and comprises an aldehyde derivatization or the introduction of a fluromethylketone or acyloxymethylketone group.

7. The method of claim 3, wherein the caspase-8 inhibitor comprises a sequence chosen from VAD, IETD and YVAD.

8. The method of claim 6, wherein the caspase-8 inhibitor comprises the sequence IETD-fmk.

9. The method of claim 1, wherein the caspase-8 inhibitor has a naturally occurring biological origin.

10. The method of claim 9, wherein the caspase-8 inhibitor has a viral, bacterial or eukaryotic origin.

11. A method of treating a tumor disease of the lymphatic system of a human, the method comprising administering to the human a therapeutically effective amount of a caspase-8 inhibitor, or a pharmaceutically effective salt thereof.

12. The method of claim 11, wherein the caspase-8 inhibitor has a non-biologically occurring molecular structure.

13. The method of claim 11, wherein the caspase-8 inhibitor comprises an oligopeptide or polypeptide.

14. The method of claim 13, wherein the caspase-8 inhibitor comprises a tetrapeptide or a partial sequence of a native protein.

15. The method of claim 13, wherein the molecular structure of the oligopeptide or polypeptide includes a chemical modification at the N— or C-terminus.

16. The method of claim 15, wherein the chemical modification is at the C-terminus and comprises an aldehyde derivatization or the introduction of a fluromethylketone or acyloxymethylketone group.

17. The method of claim 13, wherein the caspase-8 inhibitor comprises a sequence chosen from VAD, IETD and YVAD.

18. The method of claim 16, wherein the caspase-8 inhibitor comprises the sequence IETD-fmk.

19. The method of claim 11, wherein the caspase-8 inhibitor has a naturally occurring biological origin.

20. The method of claim 19, wherein the caspase-8 inhibitor has a viral, bacterial or eukaryotic origin.

21. A method of inhibiting the proliferation of peripheral blood lymphocytes in a human, the method comprising administering to the human, a therapeutically effective amount of a caspase-8 inhibitor, or a pharmacologically acceptable salt thereof.
